(19) United States
(12) Patent Application Publication
(10) Pub. No.: US 2017/0089885 A1
(43) Pub. Date: Mar. 30, 2017

(54) CELL LINE-BASED REDIRECTED T-CELL CYTOTOXICITY ASSAY

(71) Applicant: APTEVO RESEARCH AND DEVELOPMENT LLC, Seattle, WA (US)

(72) Inventors: John Blankenship, Seattle, WA (US); Elaine Sewell, Seattle, WA (US); Richard Lowe, Bothell, WA (US)

(21) Appl. No.: 15/126,922

(22) PCT Filed: Mar. 18, 2015
(86) PCT No.: PCT/US15/21252

§ 371 Date: Sep. 16, 2016

Related U.S. Application Data
(60) Provisional application No. 61/954,958, filed on Mar. 18, 2014.

(51) Int. Cl. G01N 33/50; C07K 16/30; C07K 16/32; C07K 16/28

(52) U.S. Cl. CPC G01N 33/5011; G01N 33/505; G01N 33/5014; C07K 16/2809; C07K 16/3069; C07K 16/2803; C07K 16/2896; C07K 16/32; G01N 2500/02; G01N 2500/10; G01N 2510/00; G01N 2333/7051; G01N 2333/70596; C07K 2317/622; C07K 2317/31; C07K 2317/732; C07K 2317/64

(57) ABSTRACT

This disclosure provides a cell-based assay for testing the potency of multispecific binding molecules which specifically bind a T-cell antigen and a target antigen for redirected T-cell-mediated cellular cytotoxicity. The assay uses the TALL-104 T-cell line as effector cells, and provides a sensitive, specific, and reproducible method for ensuring that purity, activity, and stability of multispecific binding molecule batches can be measured for development, clinical trials, and commercial marketing.

### CROSS REFERENCE TO RELATED APPLICATIONS

This Application claims the benefit of priority to U.S. Provisional Application No. 61/954,958, filed Mach 18, 2014, the entire contents of which are hereby incorporated by reference in their entirety for all purposes.

### BACKGROUND

A variety of bispecific antibody therapeutics are in development that can redirect T-cell cytotoxicity (or RTCC) towards tumor cells (or other target cells of interest) by cross-linking the T-cell receptor complex on the surface of T-cells with tumor specific antigens (or other target antigens of interest) expressed on the tumor or other target cell. A schematic is shown in FIG. 1. Multispecific binding molecules that bind selectively to T-cells and tumor cells offer a mechanism to redirect T-cell cytotoxicity towards the tumor cells and treatment of cancer.

Bispecific binding molecules with compelling in vitro and in vivo RTCC activity have been developed, see, e.g., PCT Publ. No. WO 2012/145714, published Oct. 26, 2012, which describes multispecific binding molecules that bind to prostate-specific membrane antigen (PSMA) and CD3. For the development of a manufacturing process for such molecules, and for clinical development, it is desirable to have a sensitive, specific, and reproducible assay that measures the potency or activity, e.g., cytotoxic potential, of the multispecific binding molecule. Multispecific binding molecules which specifically bind effector T-cells for the purpose of RTCC have no significant direct RTCC activity in the absence of binding both the effector T-cell and the target cell, e.g., through specific binding to a target antigen of interest on the surface of a cell. A cell-based assay is used to directly measure potency.

For the purposes of monitoring redirected T-cell cytotoxicity (RTCC) induced by T-cell-specific multispecific binding molecules, researchers have tried to use freshly isolated T-cells from donors. Unfortunately, initial RTCC activity in a cytotoxic assay using freshly isolated T-cells from peripheral blood can vary significantly between donors. This significantly complicates developing a reproducible cell-based cytotoxicity assay.

What would be ideal would be an immortalized, differentiated (not CD4⁺ and CD8⁺) T-cell line with an intact T-cell receptor complex, cytotoxic capacity that showed selective RTCC activity in the presence of a multispecific binding molecule of interest. Most T-cell lines, however, are derived from leukemias originating from immature T-cells, and have either an incomplete or malfunctioning T-cell receptor complex, and/or no cytotoxic capacity. Prior to this disclosure, the inventors are not aware of any generic RTCC cell based assays with human T-cell lines, either described in the literature or commercially available. Thus, there remains a need in the art for such an assay.

This disclosure provides an assay to measure the cytotoxic potential of multispecific binding molecules using as an effector T-cell line, TALL-104 cells, available from the American Type Culture Collection (Catalog no. CRL-11386). Methods are provided herein to control cell culture and handling, and to choose an appropriate target cell, e.g., a tumor cell or a recombinant cell expressing a target tumor antigen of interest. The resulting assay has high reproducibility and excellent sensitivity. This assay is able to be used with a wide variety of target antigens and target cells of interest, and as such has the potential for use as a general release assay for multispecific binding molecules for redirected T-cell cytotoxicity.

### BRIEF SUMMARY

This disclosure provides a method of measuring the cytotoxic potential of a multispecific binding molecule comprising: a. contacting the multispecific binding molecule with an immortalized cytotoxic T-cell (CTL) effector cell, wherein the CTL effector cell is a TALL-104 cell (ATCC CRL 11386), and wherein the multispecific binding molecule specifically binds the CTL effector cell via a T-cell (TC) antigen binding domain; b. contacting the multispecific binding molecule with a target cell, wherein the target cell expresses a target antigen, and wherein the multispecific binding molecule specifically binds to the target cell via a target antigen binding domain; and c. determining the extent of specific target cell death upon formation of an effector cell-multispecific binding molecule-target cell complex, thereby determining the cytotoxic potential of the multispecific binding molecule.

In certain aspects of the method, the multispecific binding molecule is a recombinant polypeptide which comprises a TC antigen binding domain and a target antigen binding domain.

In certain aspects a TC antigen binding domain specifically binds an antigen of the human T-cell receptor (TCR) complex, e.g., TCRα, TCRβ, CD3γ, CD3δ, CD3ε, or a combination thereof. In certain aspects a TC antigen binding domain comprises the antigen-binding region of an antibody, or an antigen-binding fragment, variant, or derivative thereof, e.g., the TC antigen binding domain can comprises: 6 CDRs of an antibody variable region, the VH and VL of an antibody, or a combination thereof. In certain aspects, the TC antigen binding domain is a scFv.

In certain aspects the target antigen binding domain specifically binds a target antigen of interest, or a fragment thereof comprising at least an epitope of the target antigen. In certain aspects the target antigen comprises an antigen derived from a cancer cell or tumor cell. In certain aspects the target antigen is a tumor-specific antigen. In certain aspects the target antigen comprises an amino acid sequence derived from one or more of CD123, gpA33, EpCAM (epithelial cell adhesion molecule), CD19, CD20, RON, PSMA, CD37, Her2, CLEC12A, CD33 or CEA.

In certain aspects, the target antigen binding domain comprises the antigen-binding region of an antibody, or an antigen-binding fragment, variant, or derivative thereof, e.g., the target antigen binding domain can comprise the 6 CDRs of an antibody variable region, the VH and VL of an antibody, or a combination thereof. In certain aspects the target antigen binding domain is a scFv.

In certain aspects of the method, the target cell expresses the target antigen on its surface, e.g., the target cell can naturally express the target antigen on its surface, or the target cell can be a recombinant cell engineered to express the target antigen on its surface. In certain aspects the target antigen is preferentially or exclusively expressed on cancerous cells or tumor cells.

In various aspects of the method, the multispecific binding molecule is first contacted with the target cell and then contacted with the effector cell, the multispecific binding molecule is first contacted with the effector cell and then contacted with the target cell, or the multispecific binding molecule is contacted with the effector cell and the target cell simultaneously.

In certain aspects of the method, the ratio of effector cells to target cells ranges from about 50:1 to about 1:1, e.g., from about 10:1 to about 3:1, e.g., about 10:1, about 5:1, or about 3:1.

In certain aspects of the method, target cell death is measured by determining specific target cell lysis, e.g., the target cells can be labeled with chromium-51 (⁵¹Cr), and lysis can be measured as ⁵¹Cr release. In certain aspects of the method, target cell death is measured by incorporating a marker of apoptosis or cell death into the target cells, for example the marker can be 7-amino-actinomycin D or annexin V. In certain aspects of the method, target cell death is measured by detection of caspase activation in target cells, detection of Granzyme B release by TALL-104 cells via an ELISPOT assay, or CD107a mobilization to the cell surface of TALL-104 effector cells. In certain aspects of the method, target cell death is measured by counting surviving cells, for example, the target cells can be modified to express intracellularly a fluorescent protein or a luminescent protein or a reporter/detectable protein, such as an enzyme, e.g., luciferase, β-galactosidase, etc.

In various aspects of the method, the cytotoxic potential of the multispecific binding molecule can be measured qualitatively or quantitatively.

In certain aspects of the method, the TALL-104 cells are provided as a frozen aliquot, and are used in the method immediately upon thawing.

Further provided in this disclosure is a method of testing the potency of a multispecific binding molecule batch, where the method comprises assaying the multispecific binding molecule batch according to the assays and methods provided herein, where potency correlates with cytotoxic potential. For example, the assays and methods provided herein can identify multispecific binding molecule batches that are contaminated, degraded, fragmented, improperly folded, or any combination thereof. Moreover, potency can be tested to validate a manufacturing process, a batch of the multispecific binding molecule, or a lot of the multispecific binding molecule.

Further provided in this disclosure is a method of determining susceptibility of a target cell to lysis by a multispecific binding molecule, where the method comprises testing the target cell according to the assays and methods provided herein, using a multispecific binding molecule of predetermined specificity for the first antigen-binding domain of interest.

Further provided in this disclosure is a method of screening (e.g., a library of) multispecific binding molecules for target specificity, ‘T’-cell specificity, or both, where the method comprises testing the molecule or library using the methods and assays provided herein, and identifying one or more multispecific binding molecules with cytotoxic potential.

Further provided in this disclosure is a kit for performing the methods and assays provided herein, where the kit includes one or more vials of frozen TALL-104 cells. The kit can also include one or more reagents, assay plates, and experimental methods, or one or more vials of frozen target cells.

### BRIEF DESCRIPTION OF THE DRAWINGS/FIGURES

FIG. 1 shows a schematic of redirected T-cell cytotoxicity (RTCC) using a multispecific binding molecule which specifically binds a T-cell antigen, e.g., a T-cell receptor antigen, and also specifically binds to a target antigen of interest, expressed on a target cell of interest.

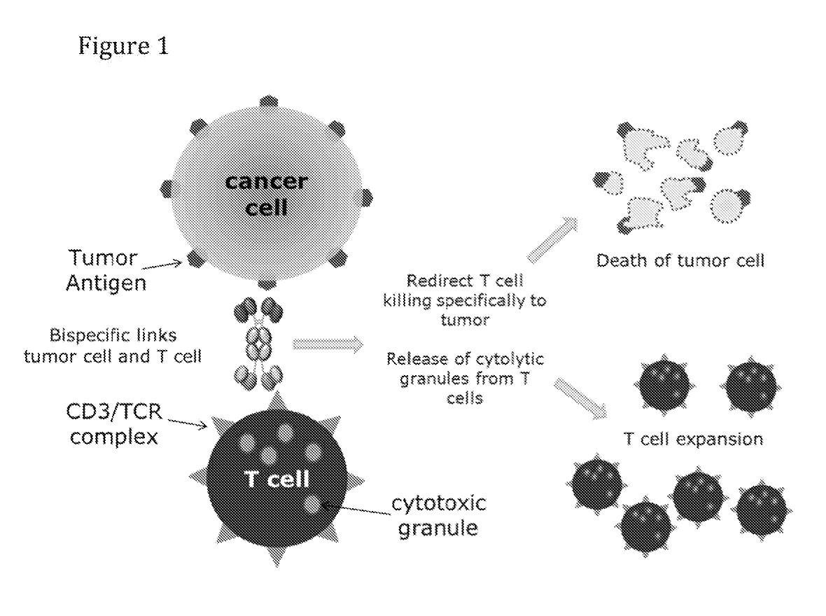

FIG. 2 shows RTCC activity of a PSMA x CD3 bispecific binding molecule on MDA-PCa-2b cells (PSMA⁺) with frozen TALL-104 cells, using a ⁵¹Cr release assay at 4 hours.

FIG. 3 shows RTCC activity of CD19 x CD3 and CD37 x CD3 bispecific binding molecules using frozen TALL-104 effector cells and Ramos target cells (CD19⁺, CD37⁺), using a ⁵¹Cr release assay at 4 hours.

FIG. 4A shows RTCC activity of a PSMA x CD3 bispecific binding molecule on MDA-PCa-2b cells with fresh or frozen TALL-104 cells, using a ⁵¹Cr release assay at 4 hours.

FIG. 4B compares the RTCC activity of a PSMA x CD3 bispecific binding molecule on C4-2B cells (PSMA⁺) cells with fresh or frozen TALL-104 cells, using a ⁵¹Cr release assay at 4 hours.

FIG. 5A is a schematic showing intact and partially degraded PSMA x CD3 bispecific binding molecules tested in the RTCC assay.

FIG. 5B compares the RTCC activity of an intact PSMA x CD3 bispecific binding molecule with the activity of two partially degraded PSMA x CD3 bispecific binding molecules (shown in FIG. 5A) on MDA-PCa-2b cells with frozen TALL-104 cells, using a ⁵¹Cr release assay at 4 hours.

FIG. 6 illustrates RTCC activity of Her2xCD3 and CD37xCD3 bispecifics using frozen TALL-104 cells and BT474 cells.

### DETAILED DESCRIPTION

In this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Wherever embodiments are described herein with the language “comprising,” otherwise analogous embodiments described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the disclosure, which can be had by reference to the specification as a whole. Accordingly, the terms defined immediately below are more fully defined by reference to the specification in its entirety.

“Polypeptide,” “peptide” and “protein” are used interchangeably and refer to a polymeric compound comprised of covalently linked amino acid residues.

### T Lymphocytes

T lymphocytes, or T-cells are a heterogeneous group of lymphocytes that can be differentiated into two large subsets by expression of the CD4 (so-called “helper” T-cells) and CD8 (so-called “effector” T-cells) cell surface markers. Within these subsets, additional subsets can be identified representing different states of T-cell maturation and function. Peripheral T-cell populations can differ substantially between any two individuals.

“CD3” is known in the art as a multi-protein transmembrane complex of six chains (see, e.g., Abbas and Lichtman, 2003; Janeway et al., p. 172 and 178, 1999), which are subunits of the T-cell receptor (TCR) complex. In mammals, the CD3 subunits of the T-cell receptor complex are a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T-cell receptor chains. The intracellular tails of the CD3γ, CD3δ, and CD3ε chains each contain a single conserved motif known as an “immunoreceptor tyrosine-based activation motif” or ITAM, whereas each CD3ζ chain has three. It is believed the ITAMs are important for the signaling capacity of a TCR complex. CD3 as used in the present disclosure can be from various animal species, including human, monkey, mouse, rat, or other mammals.

The “T-cell receptor” or TCR is a molecule found on the surface of T-cells that, along with CD3, is generally responsible for recognizing antigens bound to major histocompatibility complex (MHC) molecules. It consists of a disulfide-linked heterodimer of the highly variable α and β chains in most T-cells. In other T-cells, an alternative receptor made up of variable γ and δ chains is expressed. Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see Abbas and Lichtman, Cellular and Molecular Immunology (5th Ed.), Editor: Saunders, Philadelphia, 2003; Janeway et al., Immunobiolog: The Immune System in Health and Disease, 4^{th} Ed., Current Biology Publications, p 148, 149, and 172, 1999). TCR as used in the present disclosure can be from various animal species, including human, mouse, rat, or other mammals.

“TCR complex,” as used herein, refers to a complex formed by the association of CD3 chains with other TCR chains. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCRδ chain. In some aspects, a TCR complex can comprise any combination of CD3γ, CD3δ, CD3ç, CD3ζ, TCRα, TCRβ, wherein the combination is capable of functioning as a TCR complex. In some aspects, a TCR complex can comprise at least one active fragment, mutant, variant, or derivative of a CD3γ, CD3δ, CD3ε, CD3ζ, TCRα, or TCRβ chain, or any combination thereof.

“A component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains). The term “a component of a TCR complex” also includes active fragments, mutants, variants, or derivatives of TCR chains (TCRα, TCRβ, TCRγ or TCRδ), active fragments, mutants, variants, or derivatives of CD3 chains (i.e., CD3γ, CD3δ, CD3ε or CD3ζ), or a complex formed by two or more TCR chains or CD3 chains wherein at least one of the chain is an active fragment, mutant, variant, or derivatives of a TCR or CD3 chain.

“Redirected T-cell cytotoxicity” and “RTCC,” as used herein, refer to a T-cell-mediated process in which a T-cell is recruited to a target cell using a multi-specific protein that is capable of specifically binding both the T-cell and the target cell, and whereby a target-directed T-cell cytotoxic response is elicited against the target cell. RTCC is applicable to any T-cells, including, but not limited to CD8⁺ or cytotoxic T-cells (CTL), CD4 or helper T-cells, regulatory T-cells (T_{reg}), and natural killer T-cells (NKT).

### TALL-104 Cells

This disclosure provides a specific, sensitive, and reproducible assay for testing the cytotoxic potential of multispecific binding molecules, e.g., multispecific antibodies, which bind to a T-cell antigen and also bind to a target cell antigen, by using TALL-104 effector cells in a cell-based cytotoxicity assay.

TALL-104 (ATCC (Cat. No. CRL-11386) is a T-cell line described in detail in U.S. Pat. No. 5,272,082. TALL-104 cells have an intact T-cell receptor complex (CD3) and are differentiated into an effector phenotype (CD8⁺). TALL-104 cells have been well characterized as having cytotoxic activity; however, this cytotoxic activity was thought to be non-specific against any tumor cell. Thus, it was unexpected that the cytotoxicity could be redirected in a selective fashion against tumor cells or other target cells of interest. Experiments showing the broad utility of TALL-104 cells in a standardized RTCC assay are disclosed herein.

While the culturing of TALL-104 cells in standard conditions can result in a background of non-specific target cell lysis in the absence of a multispecific binding molecule, the methods provided herein show specific, reproducible target cell lysis above background in the presence of a target antigen specific multispecific binding molecule. Such background lysis is not entirely detrimental to the sensitivity, specificity, and reproducibility of the assays provided herein. Nonetheless, the inventors have further developed techniques to reduce the background lysis. These techniques, e.g., using previously frozen TALL-104 cells, selection of an appropriate target cell (e.g. MDA-PCa-2b), and adjusting effector:target ratios, can reduce non-specific lysis significantly. By following this disclosure, a person of ordinary skill in the art can easily adapt these techniques for use with any T-cell-specific multispecific binding molecule and any target antigen through routine standardization.

Two prior series of experiments highlighted the difficulty of identifying and using a T-cell line as a source of effector cells for RTCC. First, a T-cell leukemia cell line (HPB-ALL, available, e.g., from Invitrogen) was tested as a possible effector cell line in RTCC assays. The HPB-ALL cell line has an intact T-cell receptor complex, but is undifferentiated (CD4⁺ CD8⁺). HPB-ALL cells showed no cytotoxic activity in RTCC assays similar to those reported in the examples below, even at very high effector to target ratios. Second, another T-cell line, Jurkat cells (e.g., ATCC # TIB-152™) has been used to study T-cell receptor signaling, and has also been reported to have cytotoxic activity. A subclone of the Jurkat cell line with high TCR expression was also tried as an effector cell in RTCC assays. Similar to HPB-ALL cells, Jurkat cells showed no cytotoxic activity in RTCC assays similar to those reported in the Examples below, even at very high effector to target ratios.

### Target Antigens

A multispecific binding molecule for use in the methods described herein comprises a T-cell (TC) antigen binding domain specific for a T-cell antigen, and a target antigen binding domain, specific for a target antigen of interest. A target antigen is typically an antigen expressed on the surface of a cell with detrimental pathology, and the multispecific binding molecule would be used as a therapy to treat a patient suffering from the detrimental pathology, e.g., cancer or other hyperproliferative disorder, or a virus infection. Accordingly, multispecific binding molecules for use in the methods provided herein have specificity for a target antigen of interest, e.g., a tumor antigen or a viral antigen. Other target antigens would be readily apparent to persons of ordinary skill in the art.

Target antigens for use in the methods provided herein can be a full-length, naturally occurring tumor antigen polypeptide, either expressed on its native tumor cell, or recombinantly on another suitable host cell. Alternatively, a target antigen for use in the methods provided herein can be a fragment, variant, or derivative of a tumor antigen, provided that the fragment, variant, or derivative can specifically interact with a target antigen binding domain on the multispecific binding molecule of interest. For example, a minimal target antigen can comprise a fragment as small as an epitope, any variant (e.g., an altered amino acid sequence) as long as the variant can cross-react with the target antigen binding domain, and can be any type of derivative, e.g., an epitope expressed in a heterologous scaffold.

In certain aspects the target antigen is a tumor antigen. Tumor antigens, including fragments, variants, and derivatives thereof comprising at least an epitope of such tumor antigens, that can be targeted with multispecific binding molecules for testing in the methods provided herein include, but are not limited to: CD123, gpA33, EpCAM (epithelial cell adhesion molecule), CD19, CD20, RON, PSMA or CD37. In certain specific embodiments the target antigen is one or more of PSMA, CD19, CD37, Her2, CLEC12A, CD33 or CEA.

### Target Cells

In order to measure the cytotoxic potential of a multispecific binding molecule according to the methods described herein, a target cell expressing a target antigen of interest on its surface is provided.

Where the target antigen is a tumor antigen or an antigen preferentially or exclusively expressed on a cancerous or hyperproliferative cells, the target cell can be the actual isolated tumor cell expressing the antigen. One or often two or more, immortalized cell lines expressing many of the exemplary target antigens listed above are readily available. Persons of ordinary skill in the art can also isolate and culture primary tumor cells.

While the methods provided herein are readily adaptable to many target cells which express target antigens of interest, for some target cells the TALL-104 cells are just too efficient at killing them non-specifically to make a workable assay. It is well within the abilities of a person of skill in the art, however, to (A) screen multiple cell lines expressing the target antigen to find one with reduced non-specific lysis and/or (B) stably transfect the target antigen of choice into a target cell line that has low background lysis to enable the assay. Methods to express a target antigen of interest in a suitable cell line include, for example lentivirus transduction, stable transfection, and the like. Suitable target cells are described elsewhere herein.

### Multispecific Binding Molecules

As used herein, the term “multispecific binding molecule,” e.g., a “multispecific antibody,” refers to a recombinant polypeptide, e.g., a recombinant antibody with the ability to specifically bind two or more antigens through two or more antigen-binding domains, e.g., antibody binding domains comprising a heavy chain variable region (VH) and a light chain variable region (VL). Exemplary multispecific binding molecules for use in the methods provided herein bind to a target antigen of interest with specificity via a target antigen binding domain and to a human TCR complex (e.g., CD3) with specificity via a T-cell (TC) binding domain. As used herein, the term “multispecific binding molecule” includes multispecific antibodies and any variants, fragments, or derivatives thereof. For example, the term includes molecules comprising functional antibody fragments or derivatives that retain binding specificity. For instance, the invention includes any polypeptide scaffold that contains variable heavy and/or light chains.

As used herein, the term “antibody” (or a fragment, variant, or derivative thereof) refers to at least the minimal portion of an antibody which is capable of binding to antigen, e.g., at least the variable domain of a heavy chain (VH) and the variable domain of a light chain (VL) in the context of a typical antibody produced by a B cell. Basic antibody structures in vertebrate systems are relatively well understood. See, e.g., Harlow et al., Antibodies: A Laboratory Manual, (Cold Spring Harbor Laboratory Press, 2nd ed. 1988). The term “antibody” is defined broadly herein, to include any polypeptide, or fragment, variant, or derivative thereof, which can specifically bind to an antigen via an antibody variable domain-like structure.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fe receptor binding, complement binding, and the like.

As indicated above, the variable regions allow a multispecific binding molecule for use in the methods provided herein to selectively recognize and specifically bind epitopes on antigens. That is, the VL domain and VH domain, or a subset of the complementarity determining regions (CDRs), of an antibody combine to form the variable region that defines a three-dimensional antigen-binding site.

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term that is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein.

Multispecific binding molecules, e.g., multispecific antibodies, or antigen-binding fragments, variants, or derivatives thereof for use in the methods provided herein include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is generally meant that a multispecific binding molecule, e.g., a multispecific antibody, or antigen-binding fragment, variant, or derivative thereof for use in the methods provided herein binds to an epitope via an antigen-binding domain, and that the binding entails some complementarity between the antigen binding domain and the epitope. According to this definition, a multispecific binding molecule, e.g., a multispecific antibody, or antigen-binding fragment, variant, or derivative thereof is said to “specifically bind” to an epitope when it binds to that epitope via its antigen-binding domain more readily than it would bind to a random, unrelated epitope.

Multispecific binding molecules, e.g., multispecific antibodies, or antigen-binding fragments, variants, or derivatives thereof for use in the methods provided herein can be described or specified in terms of the epitope(s) or portion(s) of an antigen, e.g., a TC antigen or a target antigen, that they recognize or specifically bind.

Multispecific binding molecules, e.g., multispecific antibodies, for use in the methods provided herein can recognize and bind to two or more different epitopes present on two or more different antigens (e.g., proteins) at the same time. Multispecific binding molecules for use in the methods provided herein include binding molecules which are bispecific and monovalent for each specificity, e.g., “bispecific bivalent,” binding molecules which are bispecific, monovalent for one specificity, and bivalent for the other specificity, e.g., “bispecific trivalent,” and binding molecules which are bispecific and bivalent for each specificity, e.g., “bispecific tetravalent.”

Multispecific binding molecules for use in the methods provided herein can comprise dimerized single chain polypeptides, each single chain polypeptide comprising, from amino to carboxyl terminus, a target antigen binding domain, an N-terminus linker, an immunoglobulin constant region, a C-terminus linker and a TC antigen binding domain, e.g., a CD3 binding domain. In another aspect, multispecific binding molecules for use in the methods provided herein can comprise dimerized single chain polypeptides, each single chain polypeptide comprising, from amino to carboxyl terminus, a TC antigen binding domain, e.g., a CD3 binding domain, or other binding domain that binds a T-cell antigen with specificity, an N-terminus linker, an immunoglobulin constant region, a C-terminus linker and a target antigen binding domain. In this embodiment, the N-terminus linker may comprise or may consist essentially of an immunoglobulin hinge region.

In another aspect, multispecific binding molecules for use in the methods provided herein can comprise a first binding domain linked (e.g., via a linker domain) to a second binding domain (e.g., an scFv linked via a linker to another scFv). For instance, multispecific binding molecules for use in the methods provided herein can comprise a target antigen binding domain (in VH-linker-V L or V L-linker-V H orientation) linked via a peptide linker domain to a TC antigen binding domain, e.g., a CD3 binding domain (in VH-linker-VL or VL-linker-VH orientation). A bispecific binding molecule in the scFv-linker-scFv format can comprise variable heavy and variable light domains derived from any antibody which binds to the target antigen of interest, and variable heavy and variable light domains derived from any antibody which binds to the TC antigen of interest, e.g., CD3, including, but not limited to, the variable domains disclosed herein and in the following publications: U.S. Pat. Appl. Publ. No. 2012/0034245 and PCT Publ. Nos. WO2012/145714 and WO2013/158856, each of which is incorporated herein by reference in its entirety.

In some aspects, multispecific binding molecules for use in the methods provided herein can comprise a first binding domain (e.g., an scFv) linked to a second binding domain (e.g., an scFv) by a region derived from immunoglobulins, such as an Fc, CH2, CH3 or CH2CH3 region. Exemplary multispecific binding proteins include those described in U.S. Pat. Appl. Publ. Nos. 2009/0175867 and 2012/0034245 and PCT Publ. Nos. WO 2011/121110, WO 2010/037836, WO2012/145714 and WO2013/158856, each of which is incorporated herein by reference in its entirety, or constructed in a same or similar format.

In another aspect, multispecific binding molecules for use in the methods provided herein can comprise scFv dimers or diabodies rather than whole antibodies. Diabodies and scFv can be constructed without an Fc region, using only variable domains. Diabodies are bivalent, bispecific antibodies in which VH and VL domains are expressed on a single polypeptide chain, but using a peptide linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

In another aspect, multispecific binding molecules for use in the methods provided herein can comprise a disulfide-stabilized diabody. For instance, a multispecific binding molecule can comprise two distinct polypeptides that are coexpressed to generate a covalently linked heterodimeric complex with one binding site for each of 2 specificities. In this embodiment, each Fv is formed by the association of a VL partner on one chain with a VH partner on the second chain in a VLA-VHB (first chain) and VLB-VHA (second chain) configuration. The diabody is stabilized by either of two alternative carboxyterminal heterodimerization domains: a pairing of an amino acid sequence derived from the IgG1 upper hinge (e.g., VEPKSC) on one chain, and an amino acid sequence derived from the kappa light chain (e.g., FNRGEC) on the other, or a pairing of oppositely charged, coiled-coil domains. See, for example, Moore et al., 2011, Blood. 1 17:4542-4551. In this embodiment, a multispecific binding molecule for use in the methods provided herein can comprise a first chain with a TC antigen binding domain, e.g., a CD3 binding domain VH linked to a target antigen binding domain VL and the second chain can comprise a TC antigen binding domain, e.g., a CD3 binding domain VL linked to a target antigen binding domain VH, where the two chains are linked via a disulfide bond at the C-termini. A disulfide-stabilized diabody can be designed using variable heavy and light chains derived from known target antigen binding domains and/or known TC antigen binding domains, e.g., anti-CD3 antibody binding domains, including, for instance, the variable heavy and light chains disclosed herein and in the following publications: US Pat. Appl. Pubis. US2010/0174053, US2009/0060910, and US2007/0004909, EP Patent Appl. Publs. EP2158221 and EP1868650, and PCT Publ. Nos. WO2010/080538, WO2008/157379, and WO2006/113665, incorporated by reference herein in their entireties. See also: Konterman, R. E., Bispecific Antibodies, Springer; 2011 edition (Jul. 26, 2011), incorporated herein by reference in its entirety.

In another aspect, multispecific binding molecules for use in the methods provided herein can comprise a dual variable domain binding protein capable of binding a target antigen and a TC antigen, e.g., a TCR complex antigen, with specificity. In this embodiment, the recombinant multispecific binding molecule can comprise a polypeptide chain, wherein said polypeptide chain comprises VD1-(X1)n-VD2-C—(X2)n, wherein VD1 is a first variable domain, VD2 is a second variable domain, C is a constant domain, X1 is a linker (e.g., a polypeptide linker of about 10 to 20 amino acids in length), X2 represents an Fe region and n is 0 or 1. See, for instance, U.S. Pat. No. 8,258,268.

In another aspect, multispecific binding molecules for use in the methods provided herein can comprise one, two, three, or more polypeptide chains. For instance, a multispecific binding molecule for use in the method provided herein can comprise a first chain comprising VH1-VL2, a second chain comprising CH2-CH3-VL1-VH2 and a third chain comprising CH2-CH3. In this embodiment, the VH1 and VL1 can correspond to the VH and VL of a target antigen binding domain, and VH2 and VL2 can correspond to the VH and VL of a TC antigen binding domain, e.g., anti-CD3 (or other T-cell antigen) variable domains. Alternatively, the VH1 and VL1 can correspond to the VH and VL of a TC antigen binding domain, e.g., anti-CD3 (or other T-cell antigen), and the VH2 and VL2 can correspond to the VH and VL of a target antigen binding domain. Such binding domains include, for example, those disclosed in the following publications: PCT Publ. Nos. WO2010/080538, WO 2008/157379, and WO 2006/113665, incorporated by reference herein in their entireties. See also: Konterman, R. E., Bispecific Antibodies, Springer; 2011 edition (Jul. 26, 2011), incorporated herein by reference in its entirety.

As used herein, the term “binding domain” or “binding region” refers to the domain, region, portion, or site of a protein, polypeptide, oligopeptide, or peptide that possesses the ability to specifically recognize and bind to a target molecule, such as an antigen, ligand, receptor, substrate, or inhibitor (e.g., a TC antigen or a target antigen). Exemplary binding domains include single-chain antibody variable regions (e.g., domain antibodies, sFv, scFv, scFab), receptor ectodomains, and ligands (e.g., cytokines, chemokines). In certain embodiments, the binding domain comprises or consists of an antigen binding site (e.g., comprising a variable heavy chain sequence and variable light chain sequence or three light chain complementary determining regions (CDRs) and three heavy chain CDRs from an antibody placed into alternative framework regions (FRs) (e.g., human FRs optionally comprising one or more amino acid substitutions). A variety of assays are known for identifying binding domains that specifically bind a particular target, including Western blot, ELISA, phage display library screening, and BIACORE® interaction analysis. As used herein, a multispecific binding molecule can comprise a “TC antigen binding domain” and a “target antigen binding domain.” In certain embodiments the TC antigen binding domain can be a scFv derived from a mouse monoclonal antibody (e.g., CRIS-7) that binds to a T-cell surface antigen (e.g., CD3).

As used herein, the term “cytotoxic potential” refers to the degree to which a multispecific binding molecule will promote cytotoxicity, e.g., cell death, of a target cell by bringing a T-cell, e.g., a cytotoxic T-cell into the vicinity of the target cell. Multispecific binding molecules for use in the methods provided herein cannot induce target cell death independently; rather the multispecific binding molecule must associate with the target cell and with a T-cell, e.g., a cytotoxic T-cell for target cell death to occur. The methods provided herein allow determination of a multispecific binding molecule's potential for facilitating T-cell mediated target cell death, i.e., its “cytotoxic potential.” Cytotoxic potential can be qualitative or quantitative. A qualitative determination can be useful, for example, when it is desired to screen multispecific binding molecules in which the TC antigen binding domain is kept constant but in which a library of random potential target antigen binding domains is to be screened for those that can associate with a target cell of interest and facilitate T-cell mediated target cell death. A quantitative determination can be useful for validating the potency of a manufacturing batch of a multispecific binding molecule as compared to a benchmark potency level prior to release of the batch for clinical trials or for sale. Such determinations can be useful not only for activity, but also for product quality, e.g., the methods provided herein can be used to test batches or lots of the multispecific binding molecule of interest for the presence of contaminants, degradation, improper folding, and the like. Cytotoxic potential can be expressed as the percent of target cell death above background (e.g., without the binding molecule or with an irrelevant binding molecule), using complete target cell death as 100%, e.g., if cytotoxicity is being measured by radiolabelled target cell lysis and release of a radioactive molecule, e.g., ⁵¹Cr, the percent lysis is determined from the percent radioactivity released relative to a control sample in which the target cells are treated with detergent to completely lyse the cells. See, e.g., the examples below.

### Cell-Based Cytotoxicity Assays

Cytotoxic potential according to this disclosure is measured in an in vitro cytotoxicity assay using TALL-104 cells as the effector cells. In vitro assays to measure T-cell-mediated cytotoxicity are well known to those of ordinary skill in the art. Exemplary assay formats are described below, and also can be found, e.g., in Current Protocols in Imnmunology, Coligan et al., Eds., Green Publishing Associates and Wiley-Interscience, John Wiley and Sons, New York (1991) which is herein incorporated by reference in its entirety, including supplements.

Detection of cytotoxicity of target cells can be measured through release of a label from target cells (such as, but not limited to, chromium-51, calcein-AM, or lactate dehydrogenase), incorporation of a marker of apoptosis or cell death into target cells (such as, but not limited to 7-amino-actinomycin D or Annexin V), detection of caspase activation in target cells, detection of Granzyme B release by TALL-104 cells via an ELISPOT assay, CD107a mobilization to the cell surface of TALL-104 effector cells, or cell counting of surviving target cells. The last method can be easily accomplished by modifying target cells to express intracellularly a fluorescent protein (such as, e.g., GFP, YFP, RFP, iRFP) or a luminescent protein (such as, e.g., luciferase). Overall fluorescence or luminescence of target cells can then be used to quantify target cell survival.

Specificity, sensitivity and reproducibility of the assay can be quantified by normal assay development methods, for example, testing background killing of target cells in the absence of the multispecific binding molecule, testing reproducibility from different batches of cells, testing dependence of assay results on individual parameters like effector to target cell ratios, overall cell numbers, incubation times, and other variables that will be readily apparent to persons of ordinary skill in the art. In certain embodiments, the assay can be validated under GLP or GMP compliance.

A person of ordinary skill in the art will understand how to adjust effector to target ratio to obtain the most reproducible results. The proper effector to target ratio can vary with the chosen target cell, with the multispecific binding molecule being tested, or with the type of assay being used. In certain aspects the effector to target ratio ranges from about 50:1 to about 1:1, 50:1 to about 5:1, 50:1 to about 10:1, 50:1 to about 15:1, 50:1 to about 20:1, 50:1 to about 25:1, 50:1 to about 30:1, 50:1 to about 40:1, 40:1 to about 1:1, 30:1 to about 1:1, 25:1 to about 1:1, 20:1 to about 1:1, 15:1 to about 1:1, 10:1 to about 1:1, 5:1 to about 1:1, 40:1 to about 10:1, 40:1 to about 20:1, 30:1 to about 10:1, 20:1 to about 10:1, 30:1 to about 15:1 or 30:1 to about 20:1. In certain aspects the effector to target ratio is about 50:1, about 45:1, about 40:1, about 35:1, about 30:1, about 25:1, about 20:1, about 15:1, about 10:1, about 5:1, about 3:1, about 2:1 or about 1:1. In certain aspects the effector to target ratio is about 10:1. In certain aspects the effector to target ratio is about 5:1. In certain aspects the ratio is about 3:1.

### Uses for the RTCC Assays Provided

In certain aspects, the methods and assays provided herein are used during preclinical process development, during clinical manufacturing, and during commercial manufacturing to test the potency and interchangeability of different manufacturing batches of a multispecific binding molecule. Biomolecule manufacturing is subject to myriad variables and even the slightest change in procedure can harm the purity, activity, conformation, or even safety of product. Accordingly, it is imperative that each batch of a multispecific binding molecule that is produced be tested in a standardized cell-based assay to insure that the potency stays constant from batch to batch. Once a particular assay is developed, standardized, and validated, any combination of cells (e.g., TALL-104 cells and target cells), protocols, reagents, and consumables (e.g., assay plates) can be compiled as a kit that can be used uniformly at every manufacturing facility, and for each batch produced.

The method provided herein can also be used to compare different multispecific binding proteins, compare different batches or lots of multispecific binding proteins or to test the shelf life of a multispecific binding molecule, e.g., by performing the methods provided herein at periodic time points following manufacturing after the multispecific binding molecule has been expressed, purified, formulated, finished, and filled into suitable containers, and then stored in an appropriate environment.

The methods provided herein can also be used to screen libraries of multispecific binding molecules for new or improved molecules that bind to new target antigens or to the same target antigen with improved characteristics. Libraries of binding molecules can be produced by standard methods, e.g., by cloning variable domains into a scaffold that already comprises a TC antigen binding domain. The target antigen binding domains can be completely random, or can be modifications of an existing binding domain already known to bind to a target antigen of interest. For example, target antigen binding domains can be humanized, affinity matured, germlined, or subject to other modifications to improve, e.g., efficacy or safety.

The methods provided herein can further be used to identify suitable target cells for validated cell based assays, e.g., for manufacturing. Target cells can be screened for their level of non-specific lysis or their ability to bind to a given multispecific binding molecule. Candidate target cells can also be screened for usefulness after being modified, e.g., to express a detectable protein, or to express a new target antigen.

### Kits

Once an assay is developed, standardized, and validated according to the methods provided herein, any combination of cells (e.g., TALL-104 cells and target cells), protocols, reagents, and consumables (e.g., assay plates) can be compiled as a kit that can be used uniformly at every manufacturing facility, and for each batch produced. Where TALL-104 cells are to be used immediately upon thawing, individual vials of frozen cells can be provided, where each vial provides sufficient cells for one or more assay plates to be tested. Target cells can be provided, e.g., where an assay is already validated and is to be used in various different manufacturing sites, or the kit can include only the effector cells and the common reagents, with instructions on how to identify and optimize any given target cell.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992); Ausubel et al., Current Protocols in Molecular Biology, John Wiley and Sons, Baltimore, Md. (1989), and Current Protocols in Immunology, John Wiley & Sons, New York.

### INCORPORATION BY REFERENCE

All references, articles, publications, patents, patent publications, and patent applications cited herein are incorporated by reference in their entireties for all purposes.

### EXAMPLES

### Example 1

### Redirected T-Cell Cytotoxicity Against Prostate Cancer Cells Using TALL-104 Effector Cells

To determine the specific activity of a bispecific binding molecule for inducing target-dependent T-cell cytotoxicity, a bispecific binding molecule targeting prostate-specific membrane antigen (PSMA) and CD3 was tested in a chromium (⁵¹Cr) release assay using TALL-104 cells as effector cells. See, e.g., PCT Publ. No. WO 2012/145714, published Oct. 26, 2012, which describes multispecific binding molecules that bind to prostate-specific membrane antigen (PSMA) and CD3.

MDA-PCa-2b cells (PSMA⁺) were obtained from ATCC (Manassas, Va.) and cultured according to the protocol developed by the BC Cancer Agency (www.capcelllines.ca) in BRFF-HPCl cell culture media (Athena E S, Baltimore, Md.) supplemented with 20% FBS. TALL-104 cells were obtained from ATCC (Manassas, Va.), and cultured according to the provided protocol and frozen at −80° C. in aliquots of 10 million cells.

Cytotoxicity was assessed by a ⁵¹Cr release assay. MDA-PCa-2b cells in culture were harvested, trypsinized, resuspended in BRFF-HPCl media plus 20% FBS, and aliquoted for labeling. Approximately 2.5×10⁶ MDA-PCa-2b cells were treated with 0.125 mCi of ⁵¹Cr and incubated for 75 minutes at 37° C. After 75 minutes, cells were washed 3 times with media (BRFF-HPC-1 plus 20% FBS) and resuspended in 12.5 m L of the same media. During the labeling process, 50 μL of bispecific test molecules at 4× concentrations relative to a final desired concentration ranging from 1000 pM to 1.37 pM were added to appropriate wells of U-bottom 96-well assay plates. Then, 1 vial of 10 million TALL-104 cells was thawed, resuspended in 9 mL of BRFFl-HPCl media plus 20% FBS, centrifuged, and resuspended in media (BRFFl-HPCl media plus 20% FBS) to a concentration of 1 million TALL-104 cells/mL. Approximately 100 μL of TALL-104 cells (approximately 100,000) were added per well, into assay plates containing compound dilutions, bringing the total volume to 150 μL/well. Lastly, 50 μL of labeled target cells were dispensed per well (approximately 10,000 cells/well) to bring the effector to target cell ratio to 10:1.50 μL. of 0.4% NP-40 was added to control wells containing 100 μL of media plus 50 μL of target cells, to provide a total lysis control.

Plates were incubated for 4 hours, spun at 225×g for 3 minutes, and 25 μL of supernatant were transferred from each well to the corresponding well of a 96-well LUMAPLATE® sample plate (Perkin Elmer). Sample plates were allowed to air dry in a chemical safety hood for 18 hours, and then radioactivity was read on a Topcount scintillation counter using a standard protocol. Data were processed to express percent specific lysis for each sample according to the equation: (sample cpm minus background cpm from sample with no molecule added) divided by (total lysis cpm from NP-40 lysed sample minus background cpm). The data were fit to a 4-parameter logistic curve and graphed as concentration vs. % specific lysis using GraphPad PRISM® software.

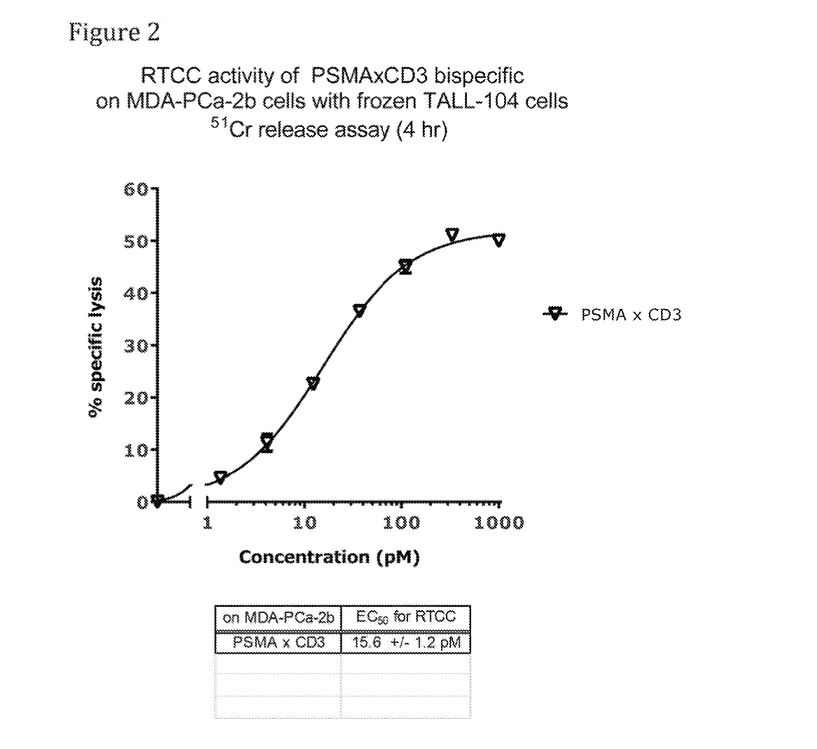

Analysis of cytotoxicity data showed strong T-cell directed cytotoxicity with the MDA-PCa-2b cells in the presence of the TALL-104 cells and the anti-PSMA directed bispecific binding molecule, reaching maximal lysis at a concentration between 100 pM and 1000 pM (FIG. 2). EC₅₀ values were calculated at 15.6 pM.

### Example 2

### Redirected T-Cell Cytotoxicity Against B-Cell Leukemias and Lymphomas Using TALL-104 Effector Cells

To compare the specific activity of different samples of bispecific polypeptide molecules directed against B-cell antigens (CD19, CD37) for inducing target-dependent T-cell cytotoxicity, samples were compared in a chromium (⁵¹Cr) release assay using TALL-104 cells as effector cells. Two different bispecific molecules were compared, the first targeting CD37 and CD3 (see, e.g., PCT Patent Application No. PCT/US14/25729, incorporated herein by reference in its entirety), and the second targeting CD19 and CD3 (see, e.g., PCT Publication No. WO2013/158856, incorporated herein by reference in its entirety).

Ramos cells (CD19⁺ CD37⁺) were obtained from ATCC (Manassas, Va.) and cultured according to the ATCC protocol in RPMI-1640 media plus 10% FBS. TALL-104 cells were obtained from ATCC (Manassas, Va.), and cultured according to the provided protocol and frozen at −80° C. in aliquots of 10 million cells.

Cytotoxicity was assessed by a ⁵¹Cr release assay. Ramos cells in culture were harvested, resuspended in RPMI-1640 media plus 10% FBS and 20 mM HEPES, and aliquoted for labeling. Approximately 2.5×10⁶ Ramos cells were treated with 0.125 mCi of ⁵¹Cr and incubated for 75 minutes at 37° C. After 75 minutes, cells were washed 3 times with media (RPMI media plus 10% FBS and 20 mM HEPES) and resuspended in 12.5 mL of the same media. During the labeling process, 50 μL of bispecific test molecules at 4× concentrations relative to final desired concentration ranging from 100 pM to 0.14 pM were added to appropriate wells of U-bottom 96 well assay plates. Then, 1 vial of 10 million TALL-104 cells was thawed, resuspended in 9 mL of RPMI media plus 10% FBS and 20 mM HEPES, centrifuged, and resuspended in media (RPMI media plus 10% FBS and 20 mM HEPES) to a concentration of 0.5 million TALL-104 cells/mL. Approximately 100 μL of TALL-104 cells (approximately 50,000) were added per well, into assay plate containing compound dilutions, bringing the total volume to 150 μL/well. Lastly, 50 μL of labeled target cells were dispensed per well (approximately 10,000 cells/well) to bring the effector to target cell ratio to 5:1.50 μL of 0.4% NP-40 was added to control wells containing 100 μL of media plus 50 μL of target cells, to provide a total lysis control.

Plates were incubated for 4 hours, spun at 225×g for 3 minutes, and 25 μL of supernatant were transferred from each well to the corresponding well of a 96-well LUMAPLATE® sample plate (Perkin Elmer). Sample plates were allowed to air dry in a chemical safety hood for 18 hours, and then radioactivity was read on a Topcount scintillation counter using a standard protocol. Data were processed to express percent specific lysis for each sample according to the equation: (sample cpm minus background cpm from sample with no molecule added) divided by (total lysis cpm from NP-40 lysed sample minus background cpm). The data were fit to a 4-parameter logistic curve and graphed as concentration vs. % specific lysis using GraphPad PRISM® software.

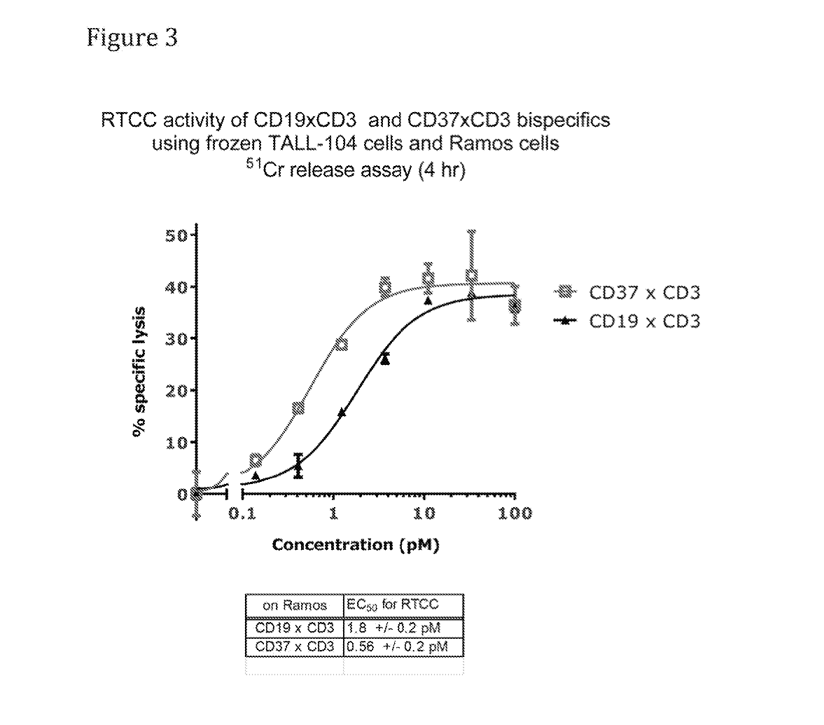

Analysis of cytotoxicity data showed strong T-cell directed cytotoxicity with the Ramos cells in the presence of the TALL-104 cells and both the anti-CD19 directed and anti-CD37 directed bispecific molecules, reaching maximal lysis at a concentration between 10 and 100 pM (FIG. 3). EC₅₀ values were calculated at 0.56 pM for the anti-CD37 directed bispecific binding molecule and 1.8 pM for the anti-CD19 directed bispecific binding molecule.

### Example 3

### A. Redirected T-Cell Cytotoxicity Against Prostate Cancer Cells Using TALL-104 Effector Cells Stored as Frozen Aliquots Compared to Cells Directly Removed from Culture Conditions

To compare the capability of fresh and frozen TALL-104 cells to perform target-dependent T-cell cytotoxicity, a bispecific binding molecule targeting PSMA and CD3 was tested in a chromium (⁵¹Cr) release assay using fresh and frozen TALL-104 cells as effector cells with prostate cancer cells as targets. See, e.g., PCT Publ. No. WO 2012/145714, published Oct. 26, 2012, which describes multispecific binding molecules that bind to prostate-specific membrane antigen (PSMA) and CD3.

MDA-PCa-2b cells (PSMA⁺) were obtained from ATCC (Manassas, Va.) and cultured according to the protocol developed by the BC Cancer Agency (www.capcelllines.ca) in 3BRFF-HPCl media (Athena E S, Baltimore, Md.) plus 20% FBS. TALL-104 cells were obtained from ATCC (Manassas, Va.), and cultured according to the provided protocol. A portion of the cell culture was frozen at −80° C. in aliquots of 10 million cells, while the remainder was maintained in culture.

Cytotoxicity was assessed by a ⁵¹Cr release assay. MDA-PCa-2b cells in culture were harvested, trypsinized, resuspended in BRFF-HPCl media plus 20% FBS, and aliquoted for labeling. Approximately 2.5×10⁶ MDA-PCa-2b cells were treated with 0.125 mCi of ⁵¹Cr and incubated for 75 minutes at 37° C. After 75 minutes, cells were washed 3 times with media (BRFF-HPC-1 plus 20% FBS) and resuspended in 12.5 mL of the same media. During the labeling process, 50 μL of bispecific test molecules at 4× concentrations relative to final desired concentration ranging from 1000 pM to 0.15 pM were added to appropriate wells of U-bottomn 96 well assay plates. For testing frozen TALL-104 cells, 1 vial of 10 million TALL-104 cells was thawed, resuspended in 9 mL of BRFFl-HPCl media plus 20% FBS, centrifuged, and resuspended in media (BRFFl-HPCl media plus 20% FBS) to a concentration of 1 million TALL-104 cells/mL. TALL-104 cells taken directly from culture conditions (fresh) were counted, centrifuged, and resuspended in media (BRFFl-HPCl media plus 20% FBS) to a concentration of 100,000 TALL-104 cells/mL. Approximately 100 μL of TALL-104 cells (approximately 100,000 frozen or 10,000 fresh) were added per well, into assay plate containing compound dilutions, bringing the total volume to 150 μL/well. Lastly, 50 μL of labeled target cells were dispensed per well (approximately 10,000 cells/well) to bring the effector to target cell ratio to 10:1 for frozen TALL-104, or 1:1 for fresh TALL-104. 50 μL of 0.4% NP-40 was added to control wells containing 100 μL of media plus 50 μL of target cells, to provide a total lysis control.

Plates were incubated for 4 hours, spun at 225×g for 3 minutes, and 25 μL of supernatant was transferred from each well to the corresponding well of a 96-well LUMAPLATE® sample plate (Perkin Elmer). Sample plates were allowed to air dry in a chemical safety hood for 18 hours, and then radioactivity was read on a Topcount scintillation counter using a standard protocol. Data. were processed to express percent specific lysis for each sample according to the equation: (sample cpm minus background cpm from sample with no molecule added) divided by (total lysis cpm from NP-40 lysed sample minus background cpm). The data were fit to a 4-parameter logistic curve and graphed as concentration vs. % specific lysis using GraphPad PRISM® software.

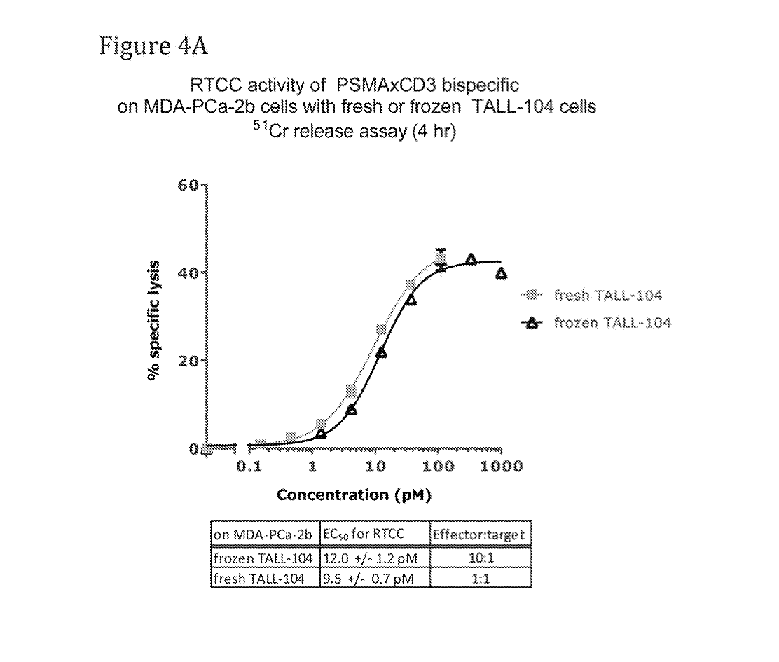

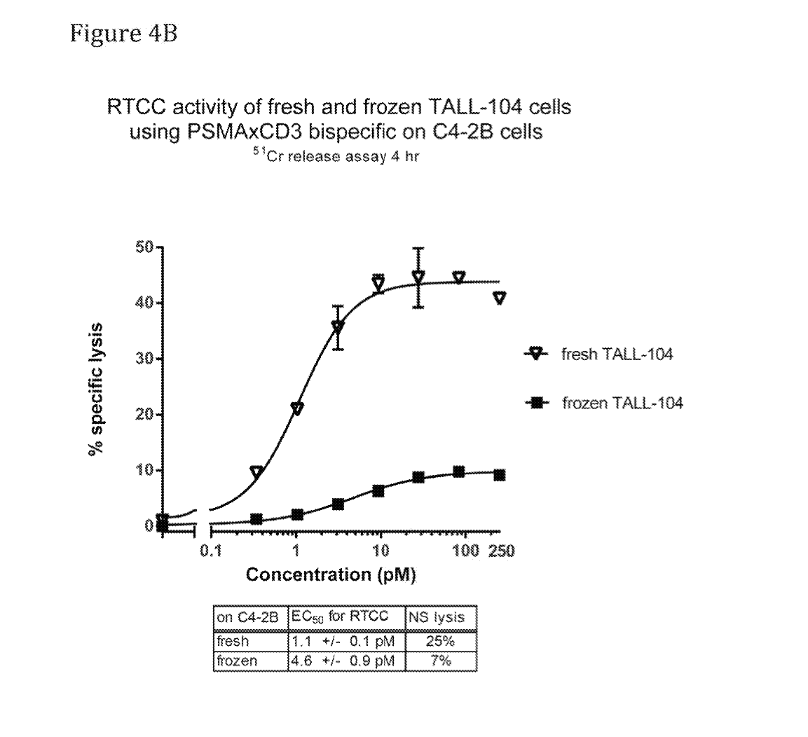

Analysis of cytotoxicity data showed strong T-cell directed cytotoxicity with the MDA-PCa-2b cells in the presence of either frozen or fresh TALL-104 cells and the anti-PSMA directed bispecific molecule, reaching maximal lysis at a concentration between 100 pM and 1000 pM (FIG. 4A). EC₅₀ values were calculated at 12.0 pM (frozen TALL-104) and 9.5 pM (fresh TALL-104).

### B. Non-Specific Lysis of Prostate Cancer Cells in Redirected T-Cell Cytotoxicity Assay is Affected by Freezing TALL-104 Effector Cells

To expand the utility of a redirected T-cell cytotoxicity assay, TALL-104 effector cells were tested in the assay after being frozen in small aliquots, as well as used directly from culture conditions. The specific activity of a bispecific binding molecule for inducing target-dependent T-cell cytotoxicity was measured using a bispecific binding molecule targeting PSMA and CD3 in a chromium (⁵¹Cr) release assay with both fresh and frozen TALL-104 cells as effector cells. See, e.g., PCT Publ. No. WO 2012/145714, published Oct. 26, 2012, which describes multispecific binding molecules that bind to prostate-specific membrane antigen (PSMA) and CD3.

C4-2B cells (PSMA⁺) were obtained from the MD Anderson Cancer Center and cultured according to published culture conditions in RPMI-1640 media (Life Technologies, Carlsbad, Calif.) plus 10% FBS. TALL-104 cells were obtained from ATCC (Manassas, V A), and cultured according to the provided protocol which supplemented the media with IL-2. Prior to use as effector cells, TALL-104 cells were grown in media without IL-2 for 3 days, divided, and one portion of TALL-104 cells were frozen at −80 C in aliquots of 10 million cells.

Cytotoxicity was assessed by a ⁵¹Cr release assay. C4-2B cells in culture were harvested, trypsinized, resuspended in RPMI-1640 media plus 10% FBS and 20 mM HEPES, and aliquoted for labeling. Approximately 2.5×10⁶ C4-2B cells were treated with 0.125 mCi of ⁵¹Cr and incubated for 75 minutes at 37° C. After 75 minutes, cells were washed 3 times with media (RPMI-1640 media plus 10% FBS and 20 mM HEPES) and resuspended in 12.5 mL of the same media. During the labeling process, 50 μL of bispecific test molecules at 4× concentrations relative to final desired concentration ranging from 250 pM to 0.01 pM were added to appropriate wells of U-bottom 96 well assay plate. TALL-104 cells in culture were centrifuged and resuspended in media (RPMI-1640 media plus 10% FBS and 20 mM HEPES) to a concentration of 300,000 TALL-104 cells/mL. To provide a second test condition, a vial of 10 million frozen TALL-104 cells was thawed, resuspended in 9 mL of RPMI-1640 media plus 10% FBS and 20 mM HEPES, centrifuged, and resuspended in media (RPMI-1640 media plus 10% FBS and 20 mM HEPES) to a concentration of 300,000 TALL-104 cells/mL. Approximately 100 μL of TALL-104 cells (approximately 30,000) was added per well, into assay plate containing compound dilutions, bringing the total volume to 150 μL/well. Lastly, 50 μL of labeled target cells were dispensed per well (approximately 10,000 cells/well) to bring the effector to target cell ratio to 3:1.50 μL of 0.4% NP-40 was added to control wells containing 100 μL of media plus 50 μL of target cells, to provide a total lysis control.

Plates were incubated for 4 hours, spun at 225×g for 3 minutes, and 25 μL of supernatant was transferred from each well to the corresponding well of a 96-well LUMAPLATE® sample plate (Perkin Elmer). Sample plates were allowed to air dry in a chemical safety hood for 18 hours, and then radioactivity was read on a Topcount scintillation counter using a standard protocol. Data were processed to express percent specific lysis for each sample according to the equation: (sample cpm minus background cpm from sample with no molecule added) divided by (total lysis cpm from NP-40 lysed sample minus background cpm). The data were fit to a 4-parameter logistic curve and graphed as concentration vs. % specific lysis using GraphPad PRISM® software.

Analysis of cytotoxicity data showed strong T-cell directed cytotoxicity with the C4-21B cells in the presence of both batches of TALL-104 cells and the anti-PSMA directed bispecific molecule, reaching maximal specific lysis at a concentration between 25 and 250 pM (FIG. 4B). Maximal specific lysis values differed between the fresh and frozen TALL-104 cells; higher levels of specific lysis were seen with the fresh TALL-104 cells (˜42%) than the frozen TALL-104 cells (˜10%). Slightly lower EC₅₀ values were also seen with the fresh TALL-104 cells (1.1 pM) than the frozen TALL-104 cells (4.6 pM). However, there was also a concomitant reduction in background non-specific lysis in the frozen TALL-104 cells (7%) compared to the fresh TALL-104 cells (25%). This suggested that freezing TALL-104 cells may provide one method to reduce non-specific lysis by TALL-104 cells and to improve the overall sensitivity of the assay.

### Example 4

### A. Reproducibility of Redirected T-Cell Cytotoxicity Assay Using TALL-104 Effector Cells

To determine the reproducibility of assay results measuring the specific activity of a bispecific binding molecule for inducing target-dependent T-cell cytotoxicity, a bispecific binding molecule targeting PSMA and CD3 was tested in a chromium (⁵¹Cr) release assay using TALL-104 cells as effector cells. See, e.g., PCT Publ. No. WO 2012/145714, published Oct. 26, 2012, which describes multispecific binding molecules that bind to prostate-specific membrane antigen (PSMA) and CD3. This assay was performed on multiple dates (assays 1 through 5 in Table 1 below), using identical frozen aliquots of the bispecific molecule and of TALL-104 cells, on the same target cell line.

MDA-PCa-2b cells (PSMA⁺) were obtained from ATCC (Manassas, Va.) and cultured according to the protocol developed by the BC Cancer Agency (www.capcelllines.ca) in BRFF-HPCl media (Athena E S, Baltimore, Md.) plus 20% FBS. TALL-104 cells were obtained from ATCC (Manassas, Va.), and cultured according to the provided protocol and frozen at −80° C. in aliquots of 10 million cells.

Cytotoxicity was assessed by a ⁵¹Cr release assay. MDA-PCa-2b cells in culture were harvested, trypsinized, resuspended in BRFF-HPCl media plus 20% FBS, and aliquoted for labeling. Approximately 2.5×10⁶ MDA-PCa-2b cells were treated with 0.125 mCi of ⁵¹Cr and incubated for 75 minutes at 37° C. After 75 minutes, cells were washed 3 times with media (BRFF-HPC-1 plus 20% FBS) and resuspended in 12.5 mL of the same media. During the labeling process, 50 μL of bispecific test molecules at 4× concentrations relative to final desired concentration ranging from 1000 pM to 1.37 pM were added to appropriate wells of U-bottom 96 well assay plate. Then 1 vial of 10 million TALL-104 cells was thawed, resuspended in 9 mL of BRFFl-HPCl media plus 20% FBS, centrifuged, and resuspended in media (BRFFl-HPCl media plus 20% FBS) to a concentration of 1 million TALL-104 cells/mL. Approximately 100 μL of TALL-104 cells (approximately 100,000) was added per well, into assay plate containing compound dilutions, bringing the total volume to 150 μL/well. Lastly, 50 μL of labeled target cells were dispensed per well (approximately 10,000 cells/well) to bring the effector to target cell ratio to 10:1.50 μL of 0.4% NP-40 was added to control wells containing 100 μL of media plus 50 μL of target cells, to provide a total lysis control.

Plates were incubated for 4 hours, spun at 225×g for 3 minutes, and 25 μL of supernatant was transferred from each well to the corresponding well of a 96-well LUMAPLATE® sample plate (Perkin Elmer). Sample plates were allowed to air dry in a chemical safety hood for 18 hours, and then radioactivity was read on a Topcount scintillation counter using a standard protocol. Data were processed to express percent specific lysis for each sample according to the equation: (sample cpm minus background cpm from sample with no molecule added) divided by (total lysis cpm from NP-40 lysed sample minus background cpm). The data were fit to a 4-parameter logistic curve and graphed as concentration vs. % specific lysis using GraphPad PRISM® software.

Analysis of cytotoxicity data from the 5 different experiments showed reproducible EC50 data with each performance of the assay. There was strong T-cell directed cytotoxicity with the MDA-PCa-2b cells in the presence of the TALL-104 cells and the anti-PSMA directed bispecific molecule, reaching maximal lysis at a concentration between 100 and 1000 pM (FIG. 2). EC₅₀ values were calculated from 13 to 17 pM, with maximum specific lysis values of 43% to 52%, and are shown in Table 1.

TABLE 1
RTCC Assay Reproducibility

| assay | EC₅₀, pM | maximum specific lysis |
|---|---|---|
| 1 | 16 | 43% |
| 2 | 16 | 51% |
| 3 | 13 | 52% |
| 4 | 17 | 50% |
| 5 | 17 | 47% |

### B. Redirected T-Cell Cytotoxicity Assay is Sensitive to Bispecific Molecule Integrity

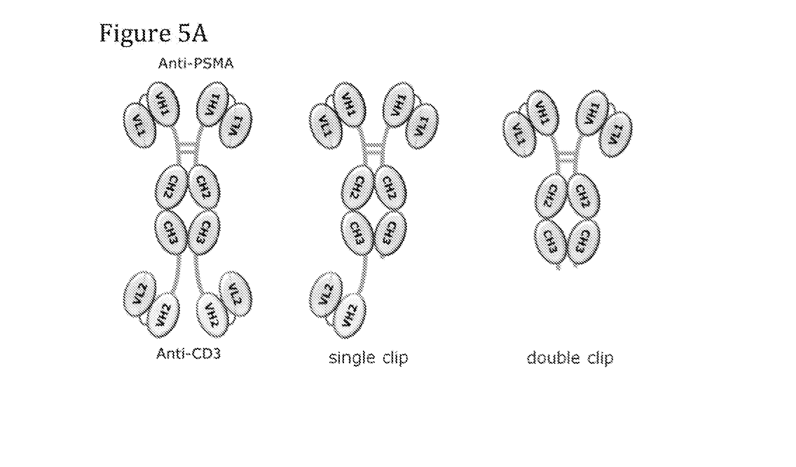

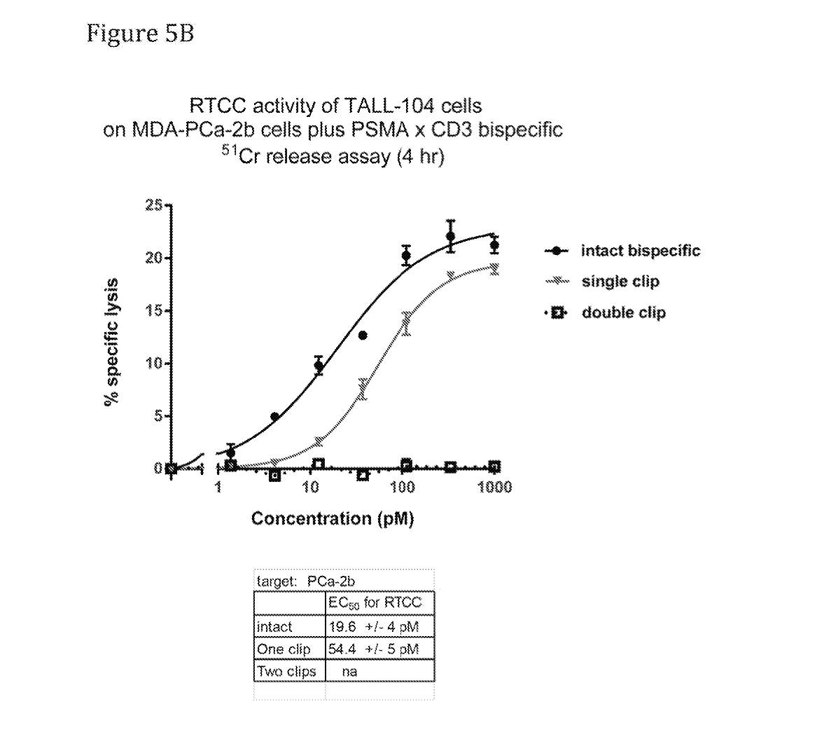

To determine the utility of the redirected T-cell cytotoxicity assay for functioning as a release assay for manufacturing purposes, the specific activity of two partially proteolyzed bispecific molecules lacking one or two CD3 binding domains (FIG. 5A) at inducing target-dependent T-cell cytotoxicity were measured and compared against intact bispecific molecule targeting PSMA and CD3 (FIG. 5A) in a chromium (⁵¹Cr) release assay using frozen TALL-104 cells as effector cells. See, e.g., PCT Publ. No. WO 2012/145714, published Oct. 26, 2012, which describes multispecific binding molecules that bind to prostate-specific membrane antigen (PSMA) and CD3.

MDA-PCa-2b cells (PSMA⁺) were obtained from ATCC (Manassas, Va.) and cultured according to the protocol developed by the BC Cancer Agency (www.capcelllines.ca) in BRFF-HPCl media (Athena E S, Baltimore, Md.) plus 20% FBS. TALL-104 cells were obtained from ATCC (Manassas, Va.), and cultured according to the provided protocol and frozen at −80 C in aliquots of 10 million cells.

Cytotoxicity was assessed by a ⁵¹Cr release assay. MDA-PCa-2b cells in culture were harvested, trypsinized, resuspended in BRFF-HPCl media +20% FBS, and aliquoted for labeling. Approximately 2.5×10⁶ MDA-PCa-2b cells were treated with 0.125 mCi of ⁵¹Cr and incubated for 75 minutes at 37° C. After 75 minutes, cells were washed 3 times with media (BRFF-HPC-1 plus 20% FBS) and resuspended in 12.5 mL of the same media. During the labeling process, 50 μL of bispecific test molecules at 4× concentrations relative to final desired concentration ranging from 1000 pM to 1.37 pM were added to appropriate wells of U-bottom 96 well assay plate. Then 1 vial of 10 million TALL-104 cells was thawed, resuspended in 9 mL. of BRFFl-HPCl media plus 20% FBS, centrifuged, and resuspended in media (BRFFl-HPCl media plus 20% FBS) to a concentration of 1 million TALL-104 cells/mL. Approximately 100 μL, of TALL-104 cells (approximately 100,000) was added per well, into assay plate containing compound dilutions, bringing the total volume to 150 μL/well. Lastly, 50 μL of labeled target cells were dispensed per well (approximately 10,000 cells/well) to bring the effector to target cell ratio to 10:1.50 μL of 0.4% NP-40 was added to control wells containing 100 μL of media plus 50 μL of target cells, to provide a total lysis control.

Plates were incubated for 4 hours, spun at 225×g for 3 minutes, and 25 μL of supernatant was transferred from each well to the corresponding well of a 96-well LUMAPLATE® sample plate (Perkin Elmer). Sample plates were allowed to air dry in a chemical safety hood for 18 hours, and then radioactivity was read on a Topcount scintillation counter using a standard protocol. Data were processed to express percent specific lysis for each sample according to the equation: (sample cpm minus background cpm from sample with no molecule added) divided by (total lysis cpm from NP-40 lysed sample minus background cpm). The data were fit to a 4-parameter logistic curve and graphed as concentration vs. % specific lysis using GraphPad PRISM® software.

Analysis of cytotoxicity data showed strong T-cell directed cytotoxicity with the MDA-PCa-2b cells in the presence of the TALL-104 cells and the intact anti-PSMA directed bispecific molecule, reaching maximal lysis at a concentration between 100 and 1000 pM (FIG. 5B). EC₅₀ values were calculated at 19.6 pM for the intact molecule. Analysis of cytotoxicity data from the partially proteolyzed molecule lacking one CD3 binding domain (FIG. 5A, 5B) showed reduced cytotoxicity compared to the intact molecule with a higher EC₅₀ (54.4 pM) and a lower level of maximal specific lysis. Analysis of data from the partially proteolyzed molecule lacking both CD3 binding domains (FIG. 5A, 5B), showed no apparent cytotoxicity, which is consistent with the mechanism of action of the bispecific molecule. This suggested that the assay was sensitive to molecular degradation, providing further justification for its use as a release assay, and an assay to measure stability.

### Example 5

### Redirected T-Cell Cytotoxicity Against Breast Cancers Using TALL-104 Effector Cells

To confirm the target-dependence of T-cell cytotoxicity initiated by TALL-104 cells, two different bispecific polypeptide molecules were compared in a chromium (⁵¹Cr) release assay measuring T-cell cytotoxicity on breast cancer cells using TALL-104 cells as effector cells. One bispecific polypeptide molecule was directed against a specific breast cancer antigen (Her2) expressed by the target cells and CD3; another was directed against a B-cell antigen (CD37) not expressed by the target cells and CD3 (see, e.g., PCT Patent Application No. PCT/US14/25729, incorporated herein by reference in its entirety).

BT-474 cells (Her2⁺ CD37⁻) were obtained from ATCC (Manassas, Va.) and cultured according to the ATCC protocol in RPMI-1640 media plus 10% FBS. TALL-104 cells were obtained from ATCC (Manassas, Va.), and cultured according to the provided protocol and frozen at −80° C. in aliquots of 10 million cells and stored in liquid nitrogen.

Cytotoxicity was assessed by a ⁵¹Cr release assay. 3BT-474 cells in culture were harvested, resuspended in RPMI-1640 media plus 10% FBS and 20 mM HEPES, and aliquoted for labeling. Approximately 1.25×10⁶ BT-474 cells were treated with 0.0625 mCi of ⁵¹Cr and incubated for 75 minutes at 37° C. After 75 minutes, cells were washed 3 times with media (RPMI media plus 10% FBS and 20 mM HEPES) and resuspended in 6.25 mL of the same media. During the labeling process, 50 μL of bispecific test molecules at 4× concentrations relative to final desired concentration ranging from 100 pM to 0.046 pM were added to appropriate wells of U-bottom 96 well assay plates. Then, 1 vial of 10 million TALL-104 cells was thawed, resuspended in 9 mL of RPMI media plus 10% FBS and 20 mM HEPES, centrifuged, and resuspended in media (RPMI media plus 10% FBS and 20 mM HEPES) to a concentration of 0.5 million TALL-104 cells/mL. Approximately 100 μL of TALL-104 cells (approximately 50,000) were added per well, into assay plate containing compound dilutions, bringing the total volume to 150 μL/well. Lastly, 50 μL of labeled target cells were dispensed per well (approximately 10,000 cells/well) to bring the effector to target cell ratio to 5:1.50 μL of 0.4% NP-40 was added to control wells containing 100 μL of media plus 50 μL of target cells, to provide a total lysis control.

Plates were incubated for 4 hours, spun at 225×g for 3 minutes, and 25 μL of supernatant were transferred from each well to the corresponding well of a 96-well LUMAPLATE® sample plate (Perkin Elmer). Sample plates were allowed to air dry in a chemical safety hood for 18 hours, and then radioactivity was read on a Topcount scintillation counter using a standard protocol. Data were processed to express percent specific lysis for each sample according to the equation: (sample cpm minus background cpm from sample with no molecule added) divided by (total lysis cpm from NP-40 lysed sample minus background cpm). The data were fit to a 4-parameter logistic curve and graphed as concentration vs. % specific lysis using GraphPad PRISM® software.

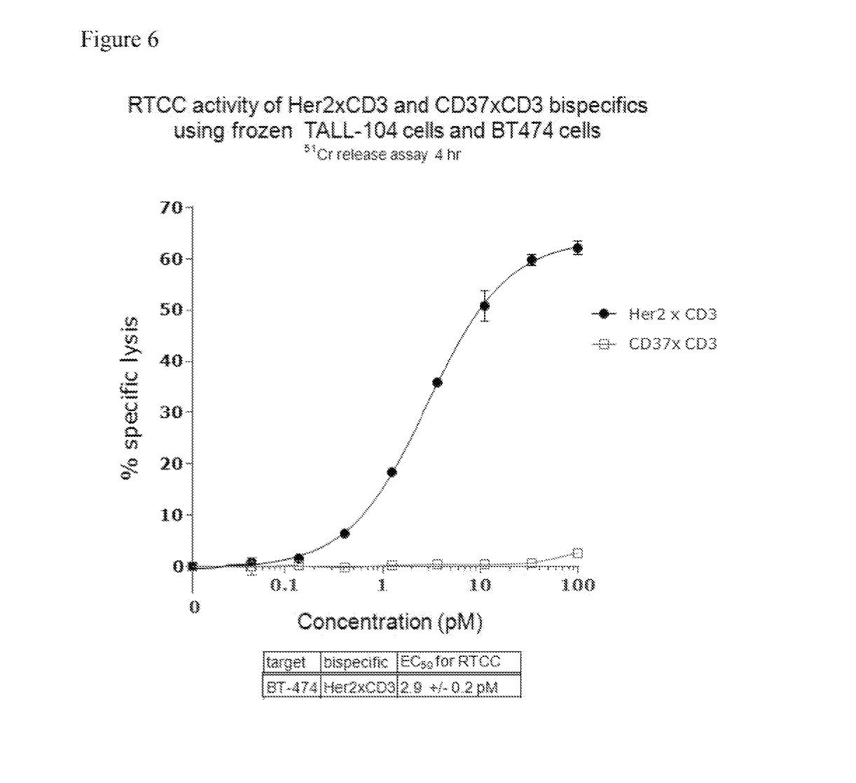

Analysis of cytotoxicity data showed strong T-cell directed cytotoxicity with the BT-474 cells in the presence of the TALL-104 cells and the anti-Her2 directed bispecific molecule, reaching maximal lysis at a concentration between 33 and 100 pM (FIG. 6). No specific lysis was observed in the presence of the anti-CD37 directed bispecific molecule, and non-specific lysis of BT-474 cells was very low (4%) in the presence of the TALL-104 cells. The EC₅₀ value was calculated at 2.9 pM for the anti-Her2 directed bispecific binding molecule.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. A method of measuring the cytotoxic potential of a multispecific binding molecule comprising:
a. contacting the multispecific binding molecule with an immortalized cytotoxic T-cell (CTL) effector cell, wherein the CTL effector cell is a TALL-104 cell (ATCC CRL 11386), and wherein the multispecific binding molecule specifically binds the CTL effector cell via a T-cell (TC) antigen binding domain;
b. contacting the multispecific binding molecule with a target cell, wherein the target cell expresses a target antigen, and wherein the multispecific binding molecule specifically binds to the target cell via a target antigen binding domain; and
c. determining the extent of specific target cell death upon formation of an effector cell-multispecific binding molecule-target cell complex, thereby determining the cytotoxic potential of the multispecific binding molecule.

2. The method of claim 1, wherein the multispecific binding molecule is a recombinant polypeptide which comprises the TC antigen binding domain and the target antigen binding domain.

3. The method of claim 1 or claim 2, wherein the TC antigen binding domain specifically binds an antigen of the human T-cell receptor (TCR) complex.

4. The method of claim 3, wherein the TC antigen binding domain specifically binds to TCRα, TCRβ, CD3γ, CD3δ, CD3ε, or a combination thereof.

5. The method of any one of claims 1 to 4, wherein the TC antigen binding domain comprises an antigen-binding region of an antibody, or an antigen-binding fragment, variant, or derivative thereof.

6. The method of claim 5, wherein the TC antigen binding domain comprises: the 6 CDRs of an antibody variable region, the VH and VL of an antibody, or a combination thereof.

7. The method of any one of claims 1 to 6, wherein the TC antigen binding domain is a scFv.

8. The method of claim 5 or claim 6, wherein the target antigen binding domain specifically binds a target antigen of interest, or a fragment thereof, wherein the fragment comprises at least an epitope of the target antigen of interest.

9. The method of claim 8, wherein the target antigen comprises an antigen derived from a cancer cell or tumor cell.

10. The method of claim 9, wherein the target antigen is a tumor-specific antigen.

11. The method of claim 10, wherein the tumor antigen comprises an amino acid sequence derived from one or more of CD123, gpA33, EpCAM (epithelial cell adhesion molecule), CD20, RON, Her2, CLEC12A, CD33 or CEA.

12. The method of claim 9, wherein the target antigen comprises one or more of CD19, CD37, or PSMA.

13. The method of any one of claims 1 to 12, wherein the target antigen binding domain comprises an antigen-binding region of an antibody, or an antigen-binding fragment, variant, or derivative thereof.

14. The method of claim 13, wherein the target antigen binding domain comprises: the 6 CDRs of an antibody variable region, the VH and VL of an antibody, or a combination thereof.

15. The method of claim 13 or claim 14, wherein the target antigen binding domain is a scFv.

16. The method of claim 1, wherein the target cell expresses the target antigen on its surface.

17. The method of claim 16, wherein the target cell naturally expresses the target antigen on its surface.

18. The method of claim 16, wherein the target cell is a recombinant cell engineered to express the target antigen.

19. The method of any one of claims 1 to 18, wherein the target antigen is preferentially or exclusively expressed on cancerous cells or tumor cells.

20. The method of any one of claims 1 to 19, wherein the multispecific binding molecule is first contacted with the target cell and then contacted with the effector cell.

21. The method of any one of claims 1 to 19, wherein the multispecific binding molecule is first contacted with the effector cell and then contacted with the target cell.

22. The method of any one of claims 1 to 19, wherein the multispecific binding molecule is contacted with the effector cell and the target cell simultaneously.

23. The method of any one of claims 1 to 22, wherein the ratio of effector cells to target cells ranges from about 50:1 to about 1:1.

24. The method of claim 23, wherein the ratio of effector cells to target cells is about 10:1, about 5:1, or about 3:1.

25. The method of any one of claims 1 to 24, wherein target cell death is measured by determining specific target cell lysis.

26. The method of claim 25, wherein the target cells are labeled with chromium⁵¹ (⁵¹Cr), and lysis is measured as ⁵¹Cr release.

27. The method of any one of claims 1 to 24, wherein target cell death is measured by incorporating a marker of apoptosis or cell death into the target cells.

28. The method of claim 27, wherein the marker is 7-amino-actinomycin 1) or annexin V.

29. The method of any one of claims 1 to 24, wherein target cell death is measured by detection of caspase activation in target cells, detection of Granzyme B release by TALL-104 cells via an ELISPOT assay, or CD107a mobilization to the cell surface of TALL-104 effector cells.

30. The method of any one of claims 1 to 24, wherein target cell death is measured by counting surviving cells.

31. The method of claim 30, wherein the target cells are modified to express intracellularly a fluorescent protein or a luminescent protein.

32. The method of any one of claims 1 to 31, wherein the cytotoxic potential of the multispecific binding molecule is measured qualitatively.

32. The method of any one of claims 1 to 31, wherein the cytotoxic potential of the multispecific binding molecule is measured quantitatively.

33. The method of any one of claims 1 to 32, wherein the TALL-104 cells are provided as a frozen aliquot, and are used immediately upon thawing.

34. A method of testing the potency of a multispecific binding molecule batch, comprising assaying the multispecific binding molecule batch according to the method of any one of claims 1 to 33, wherein potency correlates with cytotoxic potential.

35. The method of claim 34, wherein the method identifies multispecific binding molecule batches that are contaminated, degraded, fragmented, improperly folded, or any combination thereof.

36. The method of claim 34 or claim 35, wherein potency is tested to validate a manufacturing process, a batch of the multispecific binding molecule, or a lot of the multispecific binding molecule.

37. A method of determining susceptibility of a target cell to lysis by a multispecific binding molecule, comprising testing the target cell according to the method of any one of claims 1 to 33 using a multispecific binding molecule of predetermined specificity for the first antigen-binding domain of interest.

38. A method of screening multispecific binding molecules for target specificity, T-cell specificity, or both, comprising testing the molecules according to the method any one of claims 1 to 33, and identifying one or more multispecific binding molecules with cytotoxic potential.

39. A kit for performing the method of any one of claims 1 to 33, comprising one or more vials of frozen TALL-104 cells, and further comprising one or more reagents, assay plates, and experimental methods.

40. The kit of claim 39, further comprising one or more vials of frozen target cells.
